(19) United States
(12) Patent Application Publication
(10) Pub. No.: US 2020/0046521 A1
(43) Pub. Date: Feb. 13, 2020

(54) Padding with integral breast prostheses

(71) Applicant: Nicole Elisabeth Marie Mommers-McCaffrey, New Millford, CT (US)

(72) Inventor: Nicole Elisabeth Marie Mommers-McCaffrey, New Millford, CT (US)

(21) Appl. No.: 16/426,337

(22) Filed: May 30, 2019

Related U.S. Application Data
(63) Continuation-in-part of application No. 15/988,324, filed on May 24, 2018.

(51) Int. Cl. A61F 2/52; A41C 3/14; A41C 3/12

(52) U.S. Cl. CPC A61F 2/52; A41C 3/128; A41C 3/144

(57) ABSTRACT

A single length of enhanced and contoured foam padding with two, integral breast mounds for the mastectomy patient is disclosed. This single length of enhanced padding is intended to be secured into a garment or adhered to the skin to restore a natural looking breast mound appearance in clothing while offering padded comfort over the sensitive areas caused by mastectomy.

### CROSS-REFERENCES

This application claims the benefit of U.S. Provisional Patent Application No. 62/679,290 filed on Jun. 1, 2018, which is hereby incorporated by reference in its entirety.

### BACKGROUND OF THE INVENTION

### Field of the Invention

This invention relates to convenient, lightweight breast prostheses that are placed in a garment for the mastectomy patient. More particularly, this invention relates to a quick, confident and convenient way to dress with breast mounds that are securely placed into a garment or adhered to the skin for the mastectomy patient. This said enhanced padding is intended to be placed in an appropriate position within fashionable garments or adhered to the skin thus creating an instant solution for the absence of breast mounds in clothes after mastectomy surgery—all while having incorporated padding to help comfort and protect the sensitive pectoral and sternal areas caused by the mastectomy surgical procedure.

### Background

Research shows that many women have a psychological need for breast replacement after mastectomy, whether it is temporary or permanent. There are few options available today that are temporary, lightweight, flattering, comfortable, non-constricting and convenient. Women are typically offered one or more breast prostheses that are placed inside a breast pocket which is attached to the inside bodice of a garment. These garments can be heavy, unattractive, uncomfortable, constricting, inconvenient and expensive. This invention comes from the need and/or desire of wanting to restore a temporary breast mound appearance comfortably, safely, and conveniently after mastectomy while also wanting to protect the sensitive pectoral and sternal areas caused by mastectomy. Simple things like wearing a seatbelt or hugging people or playing with pets can be hurtful and uncomfortable to the sensitive mastectomy site. This invention helps to shield and protect that sensitive mastectomy site. This invention comes from the need of women's garments with integral, secure, lightweight breast prostheses that are safe, convenient, comfortable, machine washable and dryable and non-constricting. It is important for the mastectomy patient to not wear any constricting garments because of the increased risk of lymphedema after mastectomy and lymph node removal. Lymphedema is a condition of localized fluid retention in the body which can be worsened by ill-fitting elastic banded bras or garments. This invention rids the risk of any constriction when secured into a garment appropriately and intentionally or when adhered to skin. There are breast prostheses on the market today but most are single prostheses. This invention of padding offers two, aesthetically-correct bilateral breast mound prostheses within one length of padding and is lightweight, comfortable, complimentary, non-constricting, machine washable, machine dryable and more affordable than the average single breast prosthesis. This invention also offers the single length of padding with only one of the breast mounds being thicker in width and the other side being hollow to hold a natural, remaining breast for the single mastectomy patient. This invention, when securely placed within a garment or adhered to the skin will enable the wearer to simply dress with the said invention and have no need for additional breast prostheses. This invention intends to help restore the self-confidence and mental well being of a mastectomy patient or other person while being safe and comfortable. This invention is intended to offer a quick, lightweight, convenient and flattering breast mound appearance to the mastectomy patient or anyone else desiring to have an integral breast mound appearance on their garment. This invention is unique in its design in that it offers a single, fully padded piece of lightweight, contoured and enhanced foam padding with two, integral breast mounds which is secured appropriately within a garment or adhered to the skin. This invention is extremely convenient, lightweight, machine washable and machine dryable thus making it an instant, comfortable and complimentary solution for the desire to restore or gain a natural breast mound appearance in clothing. No other breast prostheses are needed to have a complete and natural looking breast mound appearance when wearing this invention. This invention is available in different sizes, thicknesses, styles and shapes to accommodate all individuals.

### SUMMARY OF THE INVENTION

This padding present invention has resolved the above drawbacks of the current mastectomy wear on the market today by providing a convenient, comfortable, easily laundered and safe alternative to the many typical breast prostheses that are offered today. This said invention, which is intended to be securely placed into a garment or adhered to the skin offers the wearer of the said invention an instant and appropriate breast mound appearance within one length of contoured and enhanced padding instead of needing two, separate breast mound prostheses. This invention is designed for the unilateral or bilateral mastectomy patient but welcome to anyone to accommodate their need and/or desire of wanting to restore or gain a breast mound appearance in clothing. This said invention will be available in many different sizes, styles and shapes but the integrity and essence of the invention will remain constant in that the said invention offers one piece of foam padding with two, integral and aesthetically-correct breast mounds.

### BRIEF DESCRIPTION OF THE DRAWINGS

FIG. 1 illustrates a front view one representation of the padding invention within one representation of a garment embodiment.

FIG. 2, FIG. 3 and FIG. 4 each illustrates a front elevation view of a various embodiment of the padding present invention thereof;

FIG. 5 illustrates a front elevation view of the outside of the padding present invention thereof;

FIG. 6 illustrates a front elevation view of the inside of the padding present invention thereof;

FIG. 7 illustrates a front elevation, cross-sectional view, bisected horizontally across the center of the padding present invention thereof;

FIG. 8 illustrates a front perspective, cross-sectional view, bisected vertically down the center of the padding present invention thereof;

FIG. 9 illustrates a front perspective, cross-sectional view, bisected vertically down the center of a breast mound within the padding present invention thereof;

FIG. 10 illustrates a rear, perspective view of the back of one embodiment of the padding present invention thereof;

FIG. 11 illustrates a front view of two, separate breast mounds and a single piece of material which can all be joined together to form the single length of contoured and enhanced padding with bilateral breast mounds present invention thereof;

FIG. 12 illustrates a front elevation view of one embodiment of the padding present invention thereof;

FIG. 13 illustrates a front, perspective view of one embodiment of the padding present invention thereof;

FIG. 14 illustrates a front, perspective view of one embodiment of the padding invention thereof and

FIG. 15 illustrates a bottom elevation view of one embodiment of the padding invention.

### DETAILED DESCRIPTION OF THE INVENTION

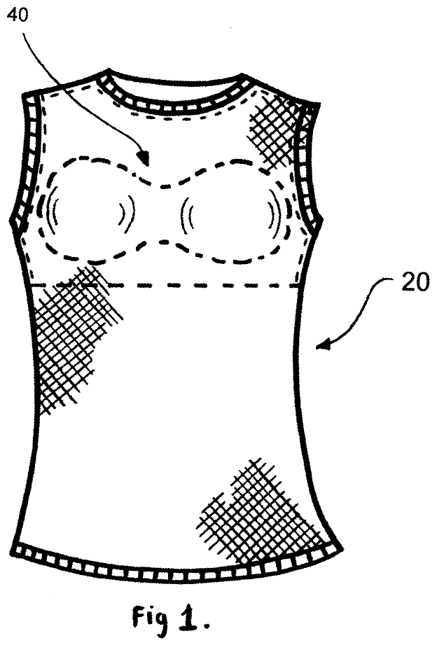

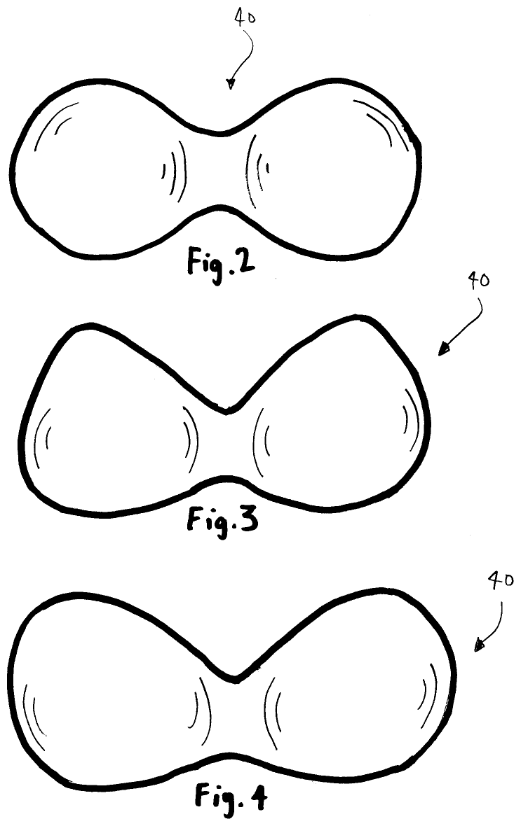

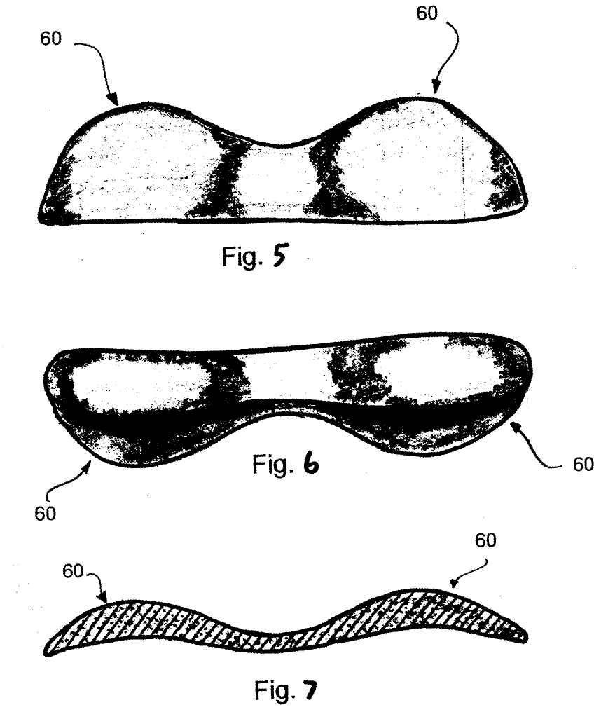

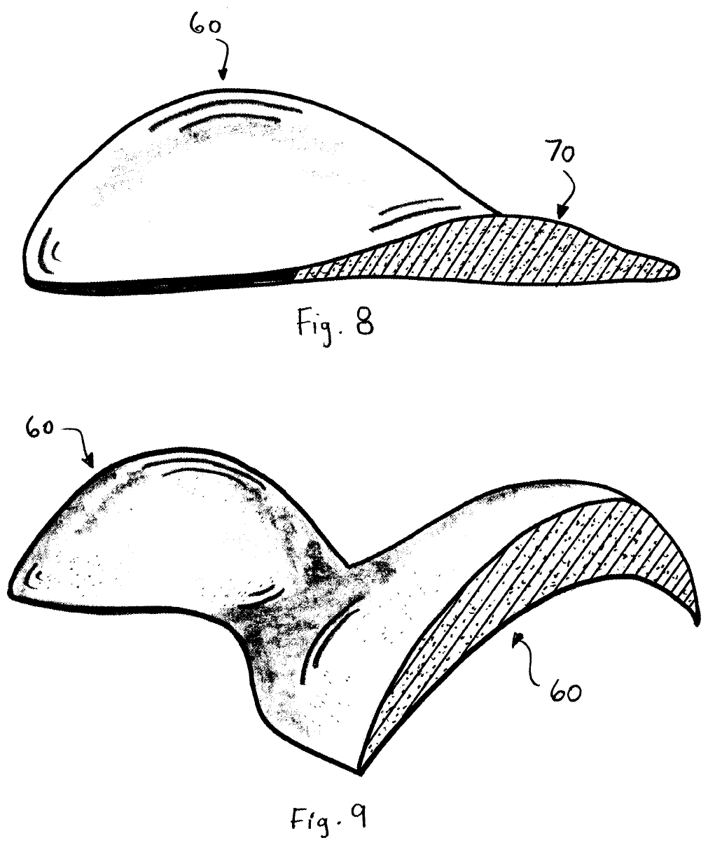

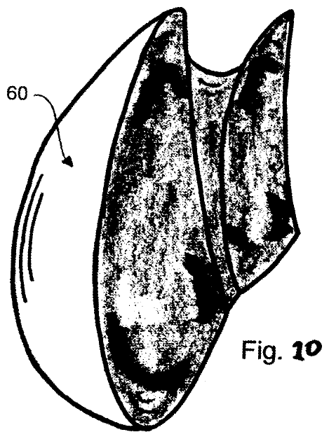

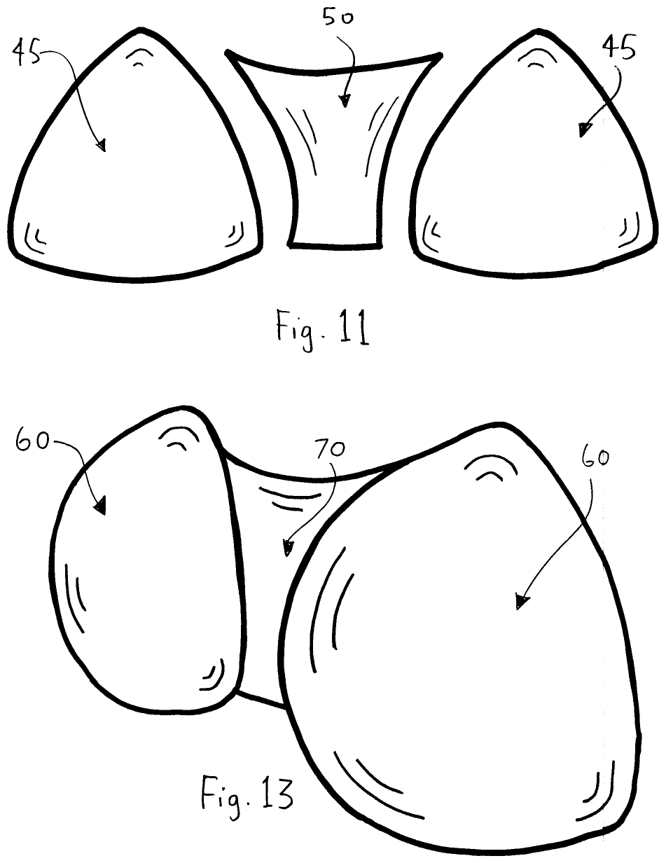

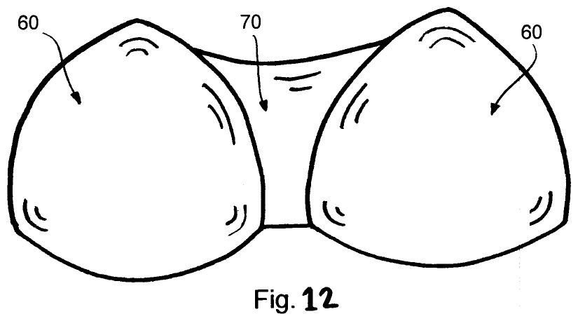

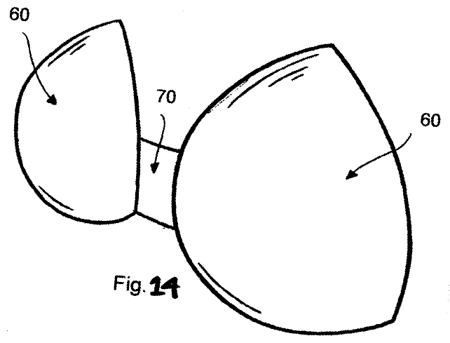

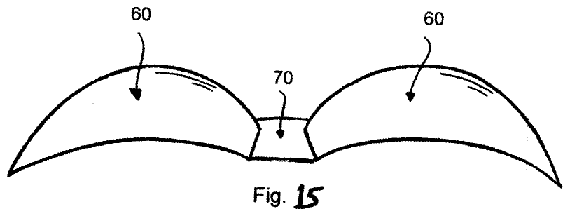

While this invention 40 is invariable in its nature and essence that of being an enhanced and contoured lightweight foam padding with two, integral and aesthetically-correct breast mound prostheses for the mastectomy patient or other, this said invention is able to take on different shapes and sizes in order to properly accommodate any and all individuals wearing this invention who are seeking to restore or gain a natural looking breast mound appearance in a quick, convenient and comfortable fashion. Samples of such different shapes are shown in FIG. 2, FIG. 3 and FIG. 4. The breast mound prostheses 60 are thicker than the typical bra cup pad making them more natural to the touch and more durable in keeping their shape. The single length of padding with two, integral and bilateral breast mounds 40 may be of any thickness and any shape. The bilateral breast mounds may be of a different thickness than each other within one length of padding in order to accommodate a persons' needs such as unilateral mastectomy. An example of one embodiment of a garment 20 with the present invention of padding with bilateral breast mounds 40 is shown in FIG. 1. The padding present invention may also be created by adding two, separate breast mounds 45 to one or more pieces of a fabric stabilizer 70 or other material as shown in FIG. 11.

What is claimed is:

1. A single length of enhanced and contoured foam padding with two integral and bilateral breast mounds prostheses for the mastectomy patient or other person.

2. According to claim 1, wherein a single length of enhanced and contoured foam padding with two integral and bilateral breast mound prostheses which is lightweight.

3. According to claim 1, wherein the said single length of enhanced and contoured foam padding with two integral and bilateral breast mound prostheses may be of any size.

4. According to claim 1, wherein the said single length of enhanced and contoured foam padding with two integral and bilateral breast mound prostheses may be of any style.

5. According to claim 1, wherein the said single length of enhanced and contoured foam padding with two integral and bilateral breast mound prostheses may be of any thickness.

6. According to claim 1, wherein the said single length of enhanced and contoured foam padding with integral and bilateral breast mound prostheses may have the two, integral bilateral breast mounds be of different thicknesses within the one length of padding in order to accommodate a unilateral mastectomy patient or other.

7. According to claim 1, wherein when the said single length of enhanced and contoured foam padding with two, integral breast mounds is not available at any time to be manufactured as one single length of padding then a final product of a single length of enhanced and contoured foam padding with two integral and bilateral breast mound prostheses may be made by attaching two breast mounds of any thickness onto a piece of fabric stabilizer or other material to create the padding present invention.
